(19) United States
(12) Patent Application Publication
(10) Pub. No.: US 2014/0171333 A1
(43) Pub. Date: Jun. 19, 2014

(54) METHOD TO OBTAIN OPTICAL MEANS ADAPTED TO A HUMAN INDIVIDUAL SUFFERING OR SUSCEPTIBLE TO SUFFER FROM ONE OR MORE GENETIC RELATED EYE DISORDER(S) OR DISEASE(S)

(71) Applicant: Laurent Alexandre, Brussels (BE)

(72) Inventor: Laurent Alexandre, Brussels (BE)

(21) Appl. No.: 13/996,867

(22) PCT Filed: Dec. 20, 2011
(86) PCT No.: PCT/EP11/73485

§ 371 Date: Jul. 31, 2013

(30) Foreign Application Priority Data
Dec. 23, 2010 (EP) 10196946.7

(51) Int. Cl. C12Q 1/68

(52) U.S. Cl. CPC C12Q 1/6883
USPC 506/9; 435/6.12; 435/6.11

(57) ABSTRACT

The present invention is related to a method comprising the step of performing a complete, partial or targeted sequencing of the genome or the epigenome of a biological sample obtained from the said individual, obtaining a genetic analysis by comparing every genetic modification present in the said sample genome or epigenome with the sequenced genome of individuals not affected by the genetic related eye disorder(s) or disease(s), and from the said previous genetic analysis, obtaining for the said individual, optical means able to prevent, correct or reduce the symptoms associated with the detected disorder(s) or disease(s).

### FIELD OF THE INVENTION

The present invention is related to a method to early obtain (design, select or manufacture) and preferentially at presymptomatic stages of a disease or syndrome affecting a human individual, optical means (i.e. lens) adapted to this human individual suffering or susceptible to suffer from one or more genetic related eye disorder(s) or disease(s), especially disorder(s) or disease(s) affecting human vision, preferably disorders or diseases over long period of time.

### BACKGROUND OF THE INVENTION

Hereditary or genetic eye disorders include conditions limited to the eye as well as ocular manifestations or other heritable disorders and complex syndromes.

Congenital cataracts (those present at birth) and retinal degenerations rank high among many genetic causes of blindness. Among the heritable retinal degenerations retinitis pigmentosa (RP), affects one person in 5000 in the United States. Onset of symptoms in retinitis pigmentosa is obtained during the first two decades of life, with progressive deterioration that leads to severe vision loss, usually by the fourth and fifth decade. The severity of the disorder varies according to the subtype, which can be transmitted by either of the two autosomal modes or in the X-linked recessive fashion.

Furthermore, an important cause of vision loss among old people is macular degeneration (Acute macular degeneration or AMD) may impact millions of adults every year. This degeneration may result in a loss (or reduction) of vision in the centre of the visual field (the macula), because of damage to the retina. The macular degeneration can make it difficult or impossible to read, recognize faces or to drive, although enough peripheral vision remains to allow other activities of daily life. When the macula is damaged, the eye loss its ability to see details and the damaged parts of the macula often cause scotomas or localized areas of vision loss.

Many of these diseases are genetically predetermined (hereditary) and there is a need to detect and treat or prevent (correct or reduce) the symptoms of these diseases or disorders in human patients at an early stage.

To the patients suffering or susceptible to suffer in the future of these diseases or disorders, there is a need to propose as early as possible in their life improved means that may prevent or treat (correct or reduce) vision loss resulting from these diseases or disorders, but also that are possibly adapted to the individual and to the evolution of his diseases or his disorders (i.e. the evolution of the symptoms or consequences (vision loss)and possibly to the geographical location, where this individual is present.

Some genetic modifications, like mutation(s) in the complement system proteins factor H (CFH), factor B (CFB), factor 3 (C3) and fibulin-5 seem to be associated with a person's risk for developing macular degeneration. Similarly, it is possible that one or more mutations in the following enzyme genes: dehydrogenase, oxygenase, acyltransferase or dismutase may be correlated with macular degeneration.

Mutation(s) in the ATP synthase gene seems to be also involved in the development of genetically determined diseases, like retinitis pigmentosa (RP).

Regular eye exams are necessary for early detection of macular degeneration, since symptoms may or may not be present in people who have the disease. This disease typically develops over a long period of time and is apparent when it has reached an advanced stage.

Furthermore, it seems that high energy visible light, especially in countries like Australia or African countries that are highly exposed to sun light, may contribute to age-related macular degeneration and other loss of vision disorders and that some of these genetic disorders or diseases are more developed in specific countries (African, Arab and South Asian countries).

### STATE OF THE ART

US 2003/0054347 discloses a method for diagnosing and treating patients at risk for eye disease, by determining and comparing polymorphisms of patients and thereafter administrating upon the patient eye topical ophthalmologic compositions, susceptible to cure the detected eye disease.

U.S. Pat. No. 4,617,299 describes ophthalmologic compositions to be topically administrated upon a patient eye.

### AIMS OF THE INVENTION

The present invention aims to improve the treatment and prevention of genetic related eye disorders or diseases affecting human vision, especially disorders or diseases affecting human vision over long periods of time and possibly according to the geographical location where this individual is present in the world.

The present invention aims in particular to early obtain (design, select or manufacture) optical means, especially improved means, preferentially at presymptomatic stages of a disease or syndrome affecting a human, these means being adapted to a human individual suffering from these genetic related (hereditary or not) eye disorders or diseases and allow to be used to prevent or treat (correct or reduce) the drawbacks (symptoms or consequences) of these diseases or disorders including their possible correlated factors, such as exposure to (sun) light, especially blue or U.V. light, preferably over long periods of time, possibly during along the full adult life of this human individual.

### SUMMARY OF THE INVENTION

A first aspect of the present invention is related to a method to obtain (design, select or manufacture) optical means (i.e. lens, able to prevent, correct or reduce symptoms associated with one or more disorder(s) or disease(s) affecting human vision) specifically adapted to a human individual suffering or susceptible to suffer from one or more genetic related eye disorder(s) or disease(s) (affecting human vision) as described in the claims.

The method (to obtain these optical means) according to the invention comprises the step of:
performing a complete or targeted sequencing of the genome or the epigenome present in a biological sample obtained from this individual,
obtaining a genetic analysis by comparing every genetic modification present in this sample genome or epigenome with the genome or epigenome of one or more (healthy) individuals (preferably at least 2, 4, 8, 10, 20, 30, 50 or 100 individuals) not affected by these genetic related eye disorder(s) or disease(s) and,
obtaining (selecting, designing or manufacturing) adequate optical means (preferably specifically selected according this previous genetic analysis) able to prevent, correct or reduce the symptoms or consequences (at least part of the vision loss and possibly correlated factors, such as sun light (preferably blue and U.V. light) associated with disorder(s) or disease(s).

Preferably, the use of these optical means by the individual may apply during a long period of time (i.e several months up to several years (such as more than 6 months, more than 1 year, more than 3 years, more than 5 years, more than 8 years or more than 10 years)) or possibly during whole adult life of an individual.

The terms “genetic related eye disorder or disease” mean one or more of the above preferred and described diseases or pathologies that have possibly a congenital origin (hereditary or not) and possibly resulting from one or more genetic (or epigenetic) modification(s), mutation(s) and/or insertion/deletion(s) in the genome of a human individual affecting human vision and possibly others diseases implicating the immune system. The terms “adequate optical means” mean lens, possibly present upon glasses and other protection elements or electronic devises for correcting layers or filter of lens against the sun light (especially against emitted light with specific wavelengths, such as U.V. light and/or blue light), including protection elements linked to the glasses (or being part of the glasses) and covering and protecting the eye (against sun light).

These lens or glasses are adapted specifically to a human individual suffering or susceptible to suffer in the future of these diseases or disorders and that can improve the daily life and reduce the generated drawbacks and consequences (i.e. at least part of the vision loss) of this individual.

The terms “sequencing of a genome” mean a high-throughput sequencing done upon the whole coding or non coding genome (including any epigenetic modification susceptible to be detected by sequencing) of an obtained sample, with an accuracy of more than 99.999% and preferably with an accuracy of more than 99.9999%. This sequencing step can be done using any technology able to generate a useful sequence for the genetic or epigenetic analysis.

“Genetic related eye disorder(s) or disease(s)” include at least disorder(s) and disease(s) affecting human vision, being hereditary or non hereditary, correlated to age or not and advantageously selected from the group consisting of acute macular degeneration (AMD) or other AMD related disorders (correlated to age or not), glaucoma, retinoschisis (RS), anisometropia, keratoconus (retinitis pigmentosa), marfan syndrome or a genetic connective tissues disorders involving a defect in chromosome 15q 21.1 affecting the synthesis of fibrillin or other diseases of genetic origin affecting vision of an individual.

In the method of the invention, optical means able to prevent, correct or reduce symptoms or consequences associated with detected disorder(s) or disease(s) are preferably selected from the group consisting of glasses or lens, including intra-ocular lens.

Advantageously, the genetic modification is any polymorphism or multigenic variation in the (individual) genome (or epigenome), preferably selected from the group consisting of mutations in the complement system proteins factor H (CFH), Factor B (CFB), factor 3 (C3), fibrulin-5 or ATP synthase genes.

Symptoms and consequences of a genetic related eye disorder or disease mean the loss of (or reduced) vision by an individual and the correlated side-effects, possibly induced by others factors, such as exposure to light, such as blue and/or U.V. light.

Advantageously, these glasses or lens are adapted for correcting individual vision according to the evolution of the disorder(s) or disease(s) and possibly of their symptoms, even not yet present at the time of detection (for instance before the appearance of a blurred vision by a patient affected by AMD or another disorder or disease).

Furthermore, in the method of the invention, these optical means, especially these glasses or lens, may comprise one or more filter(s), such as a melanin layer or any electronic device present upon or present into the lens or into the glasses and which will reduce exposure by the tested eye individual (eye human patient) to light (sunlight) by a possible modification of the layer(s) or filter(s) characteristics, especially against U.V. and/or blue light, by the use of blue blocking glasses or lens containing a yellow tint.

Preferably, these filters and/or layers are adaptable (density and/or colour modified) according to the daily light received by individual retina and/or the geographical location, where this individual is present.

The use of these optical means (i.e. lens) could be advantageously combined with the selection of a suitable diet adapted to the individual and to the detected disorder or disease, especially a diet rich in antioxidants, cartenoids, vitamins, especially vitamin C and vitamin E or minerals (copper, selenium and zinc), omega-3 fatty acids (DHA and EPA) or other drugs or treatments that could be applied for correcting or reducing the symptoms (or consequences) of the detected genetic disorder or disease of the tested individual.

The comparing (analysis or interpretation) step of the method according to the invention is performed by methods and means well known by the person skilled in the art and used for the identification of one or more genetic (epigenetic) modification(s) in the sample genome obtained from the tested individual.

Furthermore, the genetic information regarding the genome of healthy individuals, clinical data and information regarding possible location of genes involving human vision can be kept upon a data base used for the comparing (analysis or interpretation) step. An algorithm (software) could be used to perform this comparing (analysis or interpretation) step and to select or help to select (to manufacture) the most adequate optical mean for a selected individual according to its age and according to the geographical location where this individual is present.

Preferably, this biological sample is a cell, a cell extract, a tissue or a tissue extract comprising the genome (or a portion thereof) or consisting of the genome or epigenome (or a portion thereof) of the tested individual.

Preferably, the comparing step used for the genetic analysis are methods and means based upon the use of the detection of any modification of the cell genome (or epigenome) related to nucleic acids, identification with the use of any technology able to identify those modifications, like NextGenSequencing, microarrays, analysis pyrosequencing, gene expression analysis or quantitative genetic amplification (QRT-PCR, DNA fingerprinting, etc.).

Any of these techniques could be selected or combined by the person skilled in the art according to the number of genetic modifications to be detected and the number of samples to be analyzed.

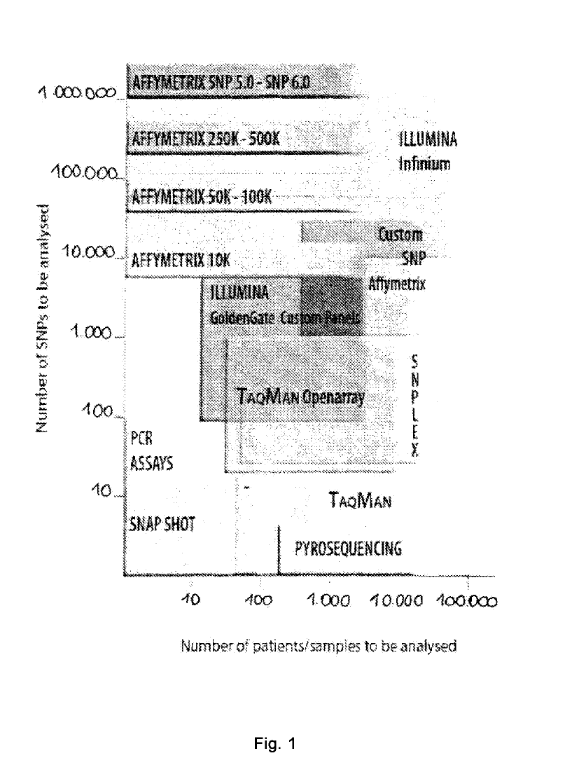

FIG. 1 shows examples of these technologies that could be selected by the person skilled in the art for such adequate genetic analysis of a biological sample.

1. A method to obtain optical means specifically adapted to a human individual suffering or susceptible to suffer from one or more genetic related eye disorder(s) or disease(s) affecting human vision and comprising the step of:
performing a complete, partial or targeted sequencing of the genome or the epigenome of a biological sample obtained from the said individual,
obtaining a genetic analysis by comparing every genetic modification present in the said sample genome or epigenome with the sequenced genome of individuals not affected by the genetic related eye disorder(s) or disease(s), and
from the said previous genetic analysis, obtaining for the said individual, optical means able to prevent, correct or reduce the symptoms associated with the detected disorder(s) or disease(s).

2. The method of claim 1, wherein the optical means are selected for preventing, correcting or reduce the symptoms associated with the detected disorder(s) or disease(s) during a period of more than 6 months.

3. The method according to the claim 2, wherein the period is the whole adult life of the individual.

4. The method according to the claim 1, wherein the optical means are selected from the group consisting of glasses or lenses.

5. The method according to the claim 4, wherein the lens are intraocular lens.

6. The method according to the claim 4, wherein the lens or glasses comprise one or more filter(s).

7. The method according to the claim 6, wherein the filter comprises a tint reducing exposure of the individual eye to light.

8. The method of claim 7, wherein the colour or density of the tint is modifiable according to exposure of the human individual eye to light and/or according to geographical location where the individual is present.

9. The method of claim 7, wherein the light is a blue light and/or UV Light and the tint is a yellow tint.

10. The method according to the claim 1, wherein the disease or disorder is selected from the group consisting of acute macular degeneration (AMD), glaucoma, retinoschisis (RS), anisometropia, keratoconus (retinitis pigmentosa), marfan syndrome or a genetic connective tissues disorders involving a defect in chromosome 15q 21.1 affecting the production of fibrillin by an individual.

11. The method according to the claim 1, wherein the comparing step comprises methods and means selected from the group consisting of epigenetic alteration, SNP genotyping, sequence methylation, mapping, sequencing, high density or low density microarray, pyrosequencing, gene expression analysis, quantitative genetic amplification and/or DNA fingerprinting.

12. The method according to the claim 1, wherein the comparing step is done upon multigenic variation genetic modification or monogenic polymorphism genetic modification.

13. The method according to the claim 1, wherein the genetic modification is selected from the group consisting of one or more mutation(s) in the complement system proteins factor H (CFH), Factor B (CFB), factor 3 (C3) and fibrulin-5 genes.

14. The method according to the claim 1, wherein the genetic modification is one or more mutation(s) in the ATP synthase gene.

15. The method according to the claim 1, wherein the biological sample is selected from the group consisting of a cell, a cell extract, a tissue or a tissue extract comprising the human individual genome.
